(19) United States
(12) Patent Application Publication
(10) Pub. No.: US 2026/0257044 A1
(43) Pub. Date: Sep. 3, 2026

(54) GUIDE WIRE

(71) Applicant: PHENOX GMBH, Bochum (DE)

(72) Inventors: Hermann MONSTADT, Bochum (DE); Volker TRÖSKEN, Witten (DE); Hans HENKES, Stuttgart (DE)

(73) Assignee: PHENOX GMBH, Bochum (DE)

(21) Appl. No.: 18/881,729

(22) PCT Filed: Jul. 7, 2023
(86) PCT No.: PCT/EP2023/068811

§ 371 Date: Jan. 6, 2025

(30) Foreign Application Priority Data
Jul. 11, 2022 (DE) 10 2022 117 162.0

(51) Int. Cl. A61M 25/09

(52) U.S. Cl. CPC A61M 25/09; A61M 2025/09083; A61M 2025/09108; A61M 2025/09133

(57) ABSTRACT

The invention relates to a guide wire (1) with a core wire (2), extending from a proximal end to a distal end, wherein a proximal portion (3) is adjacent to the proximal end and a distal portion (4) is adjacent to the distal end and the distal portion (4) of the core wire (2) is surrounded by at least one wire coil (5), wherein the wire coil (5) is composed of at least two individual wires (6), which run alongside each other in the form of a spiral, and wherein the at least two wires (6) are connected to each another at multiple connection points (7) along the wire coil (5). The guide wire (1) according to the invention is characterized by a high level of dimensional stability and improved transmission of rotational movements.

The invention relates to a medical guide wire comprising a core wire extending from a proximal end to a distal end, wherein a proximal portion is adjacent to the proximal end and a distal portion is adjacent to the distal end, and the distal portion of the core wire is surrounded by at least one wire coil.

In many medical interventions such as angioplasty or the placement of stents and flow diverters as well as endovascular examinations, guide wires are used to bring other medical devices to a target position. In particular, the guide wire paves the way within the vascular system for a catheter or balloon catheter, for example, which can be advanced over the guide wire once it has been positioned. Other medical devices can then be brought to the target position by means of or through the catheter, for example thrombectomy devices, stents, flow diverters or implants for treating aneurysms. In particular, the catheter may be a microcatheter.

In this context, the guide wire often has to be moved distally over long distances, for example from the femoral artery to intracranial blood vessels. Accordingly, a guide wire may have a length of about 1.30 to 3.50 m. One problem that arises in this context is that intracranial blood vessels are usually narrow and tortuous, which is why the guide wire must be sufficiently flexible. However, it is also important to avoid bending or even kinking the guide wire during advancement (“kink resistance”). Finally, it should also be possible to transmit torques during advancement, which prevents the guide wire from being too flexible over its entire length. A guide wire is also often specially shaped at the distal end to allow access to outgoing vessels. For example, the distal end may have a curvature or bend to the side or in the proximal direction, e.g. a 45° or 90° bend or a J-shape or a walking stick shape. Accordingly, the distal end must be malleable and retain this shape (shape retention).

For the reasons mentioned, a guide wire is therefore often made up of different portions, wherein a proximal portion is usually stiffer and less flexible to ensure the ability to advance and transmit torques, while a distal portion is designed to be very flexible in order to be able to follow narrow blood vessels. For this purpose, the proximal portion of the guide wire can be made of different materials, usually metals or alloys, than the distal portion. It is also known to reduce the outer diameter of the guide wire in the distal portion.

Furthermore, a wire coil is often fixed to the guide wire in the distal portion. The guide wire therefore has an inner core wire and a wire coil located on the core wire. The wire coil is often made of a radiopaque material in order to be able to monitor the insertion of the guide wire and, in particular, its correct positioning in the target area using X-rays. Accordingly, the wire coil can be made of a platinum or platinum-iridium wire, for example.

However, the properties of corresponding coils in the distal portion of a guide wire from the prior art are not optimal overall. This applies in particular to torsional rigidity. It is often necessary to rotate the guide wire to follow the course of a blood vessel, wherein the treating physician grasps the guide wire in a far proximal area outside the body. The rotary motion must therefore be transmitted over long distances. In the area of the wire coil attached in the distal portion, the problem arises that the wire coil, which rests against the inner wall of the vessel, can unwind or be compressed further depending on the rotational movement. The wire coil thus “stores” the rotational movement introduced, which can lead to the stored energy being released suddenly. This is undesirable, firstly because the attending physician cannot control the sudden movement, and secondly because there is a risk of injury to the vessel wall.

Furthermore, although the use of platinum or platinum alloys for the wire coil ensures sufficient X-ray visibility, the metals are not optimal in other respects. In particular, they cause undesirable stiffening of the distal portion of the guide wire, where sufficient flexibility is important in order to be able to follow the curvatures of the blood vessels.

The object of further improving guide wires known from the prior art is achieved in accordance with the invention by a guide wire with a core wire extending from a proximal end to a distal end, wherein a proximal portion is adjacent to the proximal end and a distal portion is adjacent to the distal end and the distal portion of the core wire is surrounded by at least one wire coil, wherein the wire coil is composed of at least two individual wires, which run alongside each other in the form of a spiral, and wherein the at least two wires are connected to one another at multiple connection points along the wire coil.

In the guide wire according to the invention, the wire coil is made up of at least two individual wires which run alongside each other in the form of a spiral. The wires are therefore wound parallel to each other and the wire coil is made up of at least two individual coils pushed into each other. In most cases, the wire coil is made up of two individual wires, but a structure made of 3 or more wires is also possible in principle.

Furthermore, the wires forming the wire coil and the individual coils are selectively connected to each other, namely at certain connection points. This significantly improves torsional rigidity. In particular, the disadvantageous effect described above, whereby spring energy is stored in the wire coil when rotational movements are transmitted, can be eliminated in this way.

A guide wire with a wire coil, in which the wire has several connection points along the wire coil between the turns of the wire coil, is known from U.S. Pat. No. 10,639,456. In contrast to the invention, however, these are connection points between turns of a single wire. In particular, the present invention further improves dimensional stability and the transmission of rotational movements and torques.

The core wire is the wire that primarily forms the guide wire. Said guide wire usually has a significantly larger outer diameter than the wires forming the wire coil. In particular, the wire coil can be designed such that the guide wire in the area of the wire coil has the same or only a slightly larger diameter than in more proximal located portions where only the core wire is present. In other words, the diameter of the core wire in the proximal portion, where there is no wire coil, is larger than in the distal portion with wire helix, wherein, however, the decrease in the diameter of the core wire is compensated for by the addition of the wire coil and the guide wire as a whole has a largely constant diameter. A slightly larger diameter is understood to mean a diameter that exceeds the diameter of the core wire in the proximal area by a maximum of 30%, in particular by a maximum of 20%, more preferably by a maximum of 10%. The core wire is usually of solid design, but core wires with an internal cavity or lumen are not excluded. The wire coil can be firmly connected to the core wire, advantageously by welding, wherein welding or other connection points are usually provided at the proximal and distal ends of the wire coil.

The distal portion is understood to be the area of the guide wire that adjoins the distal end of the guide wire in the proximal direction, wherein the distal end itself may or may not be part of the distal portion. The distal portion is shorter than the proximal portion and typically has a length of about 40 to 450 mm, in particular 250 to 400 mm, particularly preferably about 300 mm. A length that is not too short is advantageous in terms of torsional rigidity and preventing the wire coil from unwinding or compressing. The distal end of the guide wire itself is expediently atraumatic, in particular rounded, in order to prevent damage to vessel walls during advancement.

The proximal portion is understood to be the area of the guide wire that adjoins the proximal end of the guide wire in the distal direction, wherein the proximal end itself may or may not be part of the proximal portion. Typically, the proximal portion is considerably longer than the distal portion and makes up at least a significant part of the total length of the guide wire. Optionally, a middle portion can be arranged between the distal and proximal portions.

The terms “proximal” and “distal” are to be understood in such a manner that when inserting the guide wire, parts pointing towards the attending physician are referred to as proximal, while parts pointing away from the attending physician are referred to as distal. The guide wire is thus typically advanced through the blood vessel system in a distal direction. The term “axial” refers to the longitudinal axis of the device running from proximal to distal, the term “radial” refers to planes perpendicular thereto.

Advantageously, the core wire has a smaller outer diameter in the distal portion than in the proximal portion. This corresponds to the structure of conventional guide wires. Reducing the diameter in the distal portion ensures that the application of the wire coil does not increase the overall diameter of the guide wire too much or that it even remains largely constant. The transition between a proximal or middle portion of the core wire with a larger diameter to a distal portion with a smaller diameter is ideally made continuously via a conically shaped transition portion. However, a transition in the form of one or more stages is also possible in principle.

The wire coil can rest directly on the distal portion of the core wire, but there can also be a space between the wire coil and the distal portion of the core wire.

It makes sense for the connection points between the wires of the wire coil to be welding points. The connection of different components of a guide wire by means of welding, even when different metals come into contact with each other, is generally known from the state of the art and has proven itself. However, the use of other techniques to create the connection points, such as soldering or gluing, is not excluded. The connection between the wire coil and the core wire can, in particular, be achieved by welding, wherein alternative connection techniques such as soldering or gluing are not excluded.

According to a first advantageous embodiment, the connection points that connect the wires forming the wire coil are at least partially set in pairs, i.e. each connection point is assigned a further connection point with which it forms a pair of connection points. An offset of about 180° between the connection points of a pair of connection points has proven to be advantageous. An offset of about 180° is also understood to be an offset that deviates slightly from the ideal value of 180°, for example 5° or 10° higher or lower.

Where reference is made in this context to an offset between two connection points, this refers to the substantially round cross-section in a plane orthogonal to the longitudinal axis of the guide wire. If such a cross-section is considered to be a circle with a degree division, with 360° forming the complete circle, an offset of 180° means that the connection points are opposite each other. As the wires run in the form of a spiral, this naturally also means that there is a certain distance between the connection points in the longitudinal direction.

A subsequent pair of connection points is typically offset from the preceding pair of connection points, for example by 15° to 90°. The first connection point of the subsequent pair of connection points, viewed in the axial direction, is correspondingly offset by 15° to 90° from the first connection point of the preceding pair of connection points, and the second connection point of the subsequent pair of connection points, viewed in the axial direction, is offset by 15° to 90° from the second connection point of the preceding pair of connection points. Seen over the length of the wire coil, this results in a twist similar to the rifling of a rifled gun barrel. This ensures good flexibility of the distal portion of the guide wire. It is also possible that the offset between the pairs of connection points increases from proximal to distal, which results in a reduction in the density of connection points from proximal to distal and thus an increase in flexibility.

According to a second advantageous embodiment, the offset between the connection points of a pair of connection points is 30 to 60°, preferably about 45°. A subsequent pair of connection points is offset from the preceding pair of connection points, namely expediently by 130° to 440°. In other words, the offset between the first connection points of two consecutive pairs of connection points and between the second connection points of two consecutive pairs of connection points is 130° to 440°.

According to a particularly preferred embodiment, the offset between the pairs of connection points varies, with the offset increasing from proximal to distal. Accordingly, the distal area of the wire coil has fewer connection points than the proximal area. For example, the offset between the pairs of connection points can be 130° in the proximal area and 440° in the distal area. Accordingly, the distal area is characterized by a high level of flexibility, while the proximal area is characterized by greater rigidity and better torque transmission. The transition from a small to a large offset of the pairs of connection points can be continuous, e.g. starting with an offset of 130° up to an offset of 440°, passing through a plurality of intermediate values. However, it is also possible to provide different areas arranged one behind the other in the longitudinal direction with different offsets of the connection points, i.e. a gradual transition of the connection point density.

The above-mentioned U.S. Pat. No. 10,639,456 describes a system in which a first and a second connection point are opposite each other, whereas a third and a fourth connection point are offset by 90° with respect to the first and second connection point. In contrast to the invention, however, there are no connection points between several wires. Furthermore, the document does not show any variation of the offset that is suitable for adjusting the properties of the guide wire as desired, in particular the flexibility.

Even if no pairs of connection points are formed that are substantially opposite each other, it makes sense to provide an offset between each of the connection points, for example between 15° and 270°, in order to positively influence the flexibility of the distal portion of the guide wire.

The flexibility can be adjusted by decreasing the number of connection points between the wires along the wire coil from proximal to distal. In this way, the wire coil becomes stiffer proximally and more flexible distally, which corresponds to the object of making the guide wire as flexible as possible, in particular distally.

By using different materials for the wires, the advantageous properties of the different materials are combined. The choice of different materials for the wires forming the wire coil allows the properties of the wire coil and thus the distal portion of the guide wire to be optimally adjusted. In particular, the material of a first wire can be advantageously adjusted with regard to a desired property of the wire coil, while the material of the second wire can be advantageously adjusted with regard to another desired property.

It is advantageous to make at least one of the wires forming the wire coil radiopaque. Different metals and alloys can be used for this purpose. Examples of possible materials include platinum or platinum alloys such as a platinum-iridium alloy. Other alternatives include platinum-tungsten and platinum-nickel alloys, palladium, tantalum, gold and tungsten. It is also possible to achieve radiopacity by coating a wire with gold. Said coating can have a thickness of 1 to 6 μm, for example. Platinum-nickel alloys are advantageous due to their higher strength in order to improve formability and durability.

To increase the elasticity of the distal portion of the guide wire, it is advantageous to form at least one of the wires forming the wire coil from a superelastic or pseudoelastic alloy (shape memory alloy). Nickel-titanium alloys, such as those known as Nitinol, are particularly suitable as pseudo-elastic alloys.

Another possibility is to manufacture at least one of the wires forming the wire coil from a cobalt-chromium alloy. Cobalt-chromium alloys have advantageous properties with regard to the transmission of torques and controllability even over long distances. This is due to the high modulus of elasticity (Young's modulus) and shear modulus. A high yield strength provides good protection against kinking (“kink resistance”) and permanent bending. Cobalt-chromium-nickel alloys and cobalt-chromium-nickel-molybdenum alloys are particularly preferred. In particular, these can be largely titanium-free, which further improves the properties. A corresponding alloy is known under the name 35N LT®.

Insofar as alloys are mentioned in the context of the present invention, it is hereby clarified that the mention of metals as a component of this alloy does not exclude the possibility that the alloy contains other components. For example, a cobalt-chromium alloy can contain other components such as nickel or molybdenum in addition to cobalt and chromium. Likewise, a platinum-iridium alloy does not have to have platinum and iridium as its sole components. Insofar as metals are mentioned in the context of the present invention, this also includes alloys, wherein the alloys may also contain non-metals such as carbon or nitrogen in addition to metals. The possibilities and examples given for certain metals and alloys, such as superelastic/pseudoelastic alloys, radiopaque alloys, cobalt-chromium alloys, etc., always apply to all mentions of these alloys in the context of this description, even if they are not explicitly mentioned at a particular point, unless the text explicitly states otherwise.

The use of DFT (drawn filled tubing) wires as wires for the construction of the wire coil is also particularly preferred. DFT wires have an interior made of a certain metal and a sheath made of another metal, so that the wire combines the properties of both metals. In particular, DFT wires can have a radiopaque interior and a pseudoelastic sheath. The materials mentioned above can be used, in particular platinum alloys to achieve radiopacity and nickel-titanium alloys for pseudoelasticity.

It makes sense to make one of the wires wound next to each other radiopaque and to manufacture a second wire from a pseudo-elastic or cobalt-chromium alloy. A DFT wire is also an option for the second wire, in particular with a radiopaque interior and a sheath made of a pseudoelastic or a cobalt-chromium alloy.

It is also possible to construct at least two wires forming the wire coil from a pseudo-elastic alloy, in particular a nickel-titanium alloy. In order to achieve radiopacity, a third wire made of a radiopaque material such as a platinum alloy can be inserted between the other wires. This third wire can have a smaller cross-section than the other wires.

In order to adjust the lateral stiffness of the wire coil, the pitch of the wire coil or the individual wires forming the wire coil can be varied from proximal to distal. In particular, there may be a smaller pitch in the distal area of the wire coil than in the proximal area. The winding is correspondingly tighter distally than proximally. This can be done in portions, but an even transition is preferred, in which the pitch decreases continuously from proximal to distal.

The windings of the wires forming the wire coil can be designed such that there is a space between the wires or so that the wires are in direct contact with each other. In this way, the flexibility of the wire coil and thus also of the guide wire can be specifically adjusted. A wire coil with larger spaces tends to be more flexible than a wire coil with small, few or no spaces.

The proximal portion and any middle portion of the core wire can be at least partially made of a cobalt-chromium alloy. Since the proximal portion, optionally together with a middle portion, makes up the largest part of the length of the guide wire, this ensures that the guide wire can be easily controlled and has sufficient kink resistance. However, it is also possible to use other materials, such as stainless steel.

In the distal portion, the core wire is preferably made of a superelastic alloy, in particular a nickel-titanium alloy. As a rule, the distal portion is significantly shorter than the proximal portion and has, for example, a length of about 40 to 450 mm, in particular 250 to 400 mm, particularly preferably about 300 mm. The use of superelastic alloys in the distal portion ensures a high level of flexibility, particularly in the area where flexibility is most important.

It therefore also makes sense with regard to the core wire to design it with different portions of different flexibility, wherein the flexibility in the distal portion is typically higher than in the proximal portion. For this purpose, as described, different materials can be used in the different portions, but it is also possible to influence the flexibility via the material thickness or the cross-section of the core wire. In this case, the core wire typically has a larger cross-section proximally than distally. If different materials, in particular metals or alloys, are used in different portions, they are typically welded together at the transition points.

Furthermore, the use of DFT wires is also particularly preferred for the core wire, in particular in the distal portion. A core wire with a radiopaque interior and a superelastic sheath combines the properties of radiopacity and flexibility, which are particularly important in the distal portion. A core wire with a radiopaque interior and a sheath made of a cobalt-chromium alloy is also possible.

According to a particularly preferred embodiment, the entire or at least a large part of the core wire is made of a wire with a superelastic interior and a sheath made of a cobalt-chromium alloy or cobalt-chromium-nickel alloy. In the distal portion, the sheath is then completely or partially removed from the core wire. This can be achieved in particular by means of a grinding process. The process can be carried out to such an extent that ultimately only the superelastic interior of the wire remains in the distal portion, but this is not absolutely necessary, as the removal of part of the sheath already ensures an increase in flexibility. The advantage of this procedure is, in particular, that no dissimilar welding of different portions of the core wire is necessary.

Another advantage of a core wire consisting of a superelastic or pseudoelastic alloy such as Nitinol, at least in the distal portion, is the possibility of shaping by suitable heat treatment. The good superelastic properties ensure shape retention. This is particularly important as the distal end of a guide wire often has a curvature or bend to facilitate probing the course of blood vessels and locating branching blood vessels.

It is also possible to form one or more of the wires forming the wire coil as a coil, i.e. to twist them before the wire is then wound into the shape of the wire coil. The result is a coiled coil or double coil, which consists of a primary coil that is transformed into a secondary coil. In the wire coil, i.e. the secondary coil, several wires wound as a primary coil can lie next to each other. However, it is also conceivable to twist only one or some of the wires forming the wire coil before forming the wire coil.

Several wire coils can also be arranged radially one above the other in the distal portion of the core wire. In other words, one wire coil can be arranged further outwards with respect to another wire coil, wherein the overlap of the wire coils can be complete or partial. Guide wires with wire coils arranged one above the other are known, for example, from U.S. Pat. No. 8,480,598 B2. At least one of the wire coils is formed from at least two individual wires as described according to the invention. The overlap of the individual wire coils does not have to be complete; one or more wire coils can also be shorter than others and/or only partially overlap.

If several wire coils are arranged on top of each other, it is advantageous to wind them in opposite directions, i.e. one as a left-hand coil and one as a right-hand coil, or alternating. This also improves the transmission of torques, as differences in the direction of rotation are compensated for by the counter-rotation of the wire coils.

The at least two wires forming the wire coil do not necessarily have to run alongside each other in the form of a spiral over the entire length of the wire coil, but they should do so over most of their length, preferably over at least 70%, more preferably over at least 80% and more preferably over at least 90% of the total length of the wire coil. However, the most preferred solution is for the wires forming the wire coil to run alongside each other along the entire length of the wire coil, i.e. 100%.

At the distal end, the guide wire can have a bend or curvature. For example, the guide wire can have a bend or curvature at the distal end by a total of 45° to 180°, e.g. 90° or 135°. This gives the guide wire the shape of a J or a walking stick at the distal end. One advantage is that probing and advancing into narrow blood vessels is easier, on the one hand, because the curved or bent tip can be rotated into a position in which it follows the curvature of the blood vessel more easily. On the other hand, a guide wire with a distal bend or curvature is more atraumatic, i.e. the risk of injury to the vessel walls is reduced. Maintaining the shape of the guide wire at the distal end is helpful for fulfilling the tasks of the guide wire. The wire coil can be located entirely or partially in the area of or proximal to the curvature/bend.

In most cases, both the wires forming the wire coil and the core wire are circular in cross-section. In principle, however, other shapes are also conceivable, in particular a shape that is oval in cross-section.

The properties of the guide wire or the individual components of the guide wire can be influenced by methods known from the prior art, e.g. by cold forming, heat treatment, tempering or recrystallization annealing.

It makes sense for the guide wire to have a polymer shell on the outside. A hydrophilic coating can also be applied in addition to or instead of the polymer shell. In particular, PTFE (polytetrafluoroethylene) or another fluoropolymer can be used for the polymer shell. Other materials that can be used for the polymer shell are polyamides, polyurethanes, polyvinyl chloride, polyester, polystyrene, polyimides, polycarbonates, polyolefins such as polypropylene or polyethylene or silicones. The polymer shell and/or hydrophilic coating does not have to extend over the entire length of the guide wire; partial sheathing or coating is also possible. For example, a proximal portion may be provided with the polymer shell, while the hydrophilic coating is applied distally to improve the gliding properties of the guide wire. The hydrophilic coating can extend beyond the distal portion that carries the wire coil and can, for example, extend over a length of 30 to 40 cm. For example, polyvinylpyrrolidones, cellulose-based polymers or polyvinyl alcohols can be used as hydrophilic coatings.

The guide wire according to the invention can be used in particular in the neurovascular field, but it can also be used in the cardiovascular or peripheral field. The guide wire is particularly important for intracranial and coronary blood vessels, since in these cases, on the one hand, narrow-lumened blood vessels must be reached and, on the other hand, the guide wire has to be advanced over relatively long distances.

The diameter of the guide wire is typically in the range of 0.2 to 0.5 mm, although the diameter does not have to be constant over the entire length.

The guide wire according to the invention can be used in conjunction with OTW (over the wire) catheters, in which the guide wire runs through the entire lumen of the catheter, as well as Rx (rapid exchange) catheters. In this case, the guide wire only runs through the distal portion of the catheter, wherein the distal portion typically has a length of about 20 to 40 cm. Accordingly, the catheter has an opening proximal to the distal portion through which the guide wire passes and exits the lumen of the catheter. Such an opening is also known as a port. OTW and Rx systems are familiar to persons skilled in the art, for example, in the field of balloon catheters.

At the proximal end, the guide wire can be provided with handles to make it easier for the attending physician to handle the guide wire.

In addition to the guide wire according to the invention itself, the invention also relates to the use of the guide wire and the combination of the guide wire with other medical devices.

All descriptions of features of the invention refer in each case to all embodiments, unless the context indicates otherwise.

The invention is explained in more detail with reference to the examples of embodiments illustrated in the figures. It should be noted that the figures show preferred embodiment variants of the invention, but the invention is not limited thereto. In general, the invention comprises any combination of the technical features listed in the claims or described in the description as being relevant to the invention, insofar as this is technically feasible.

In the figures:

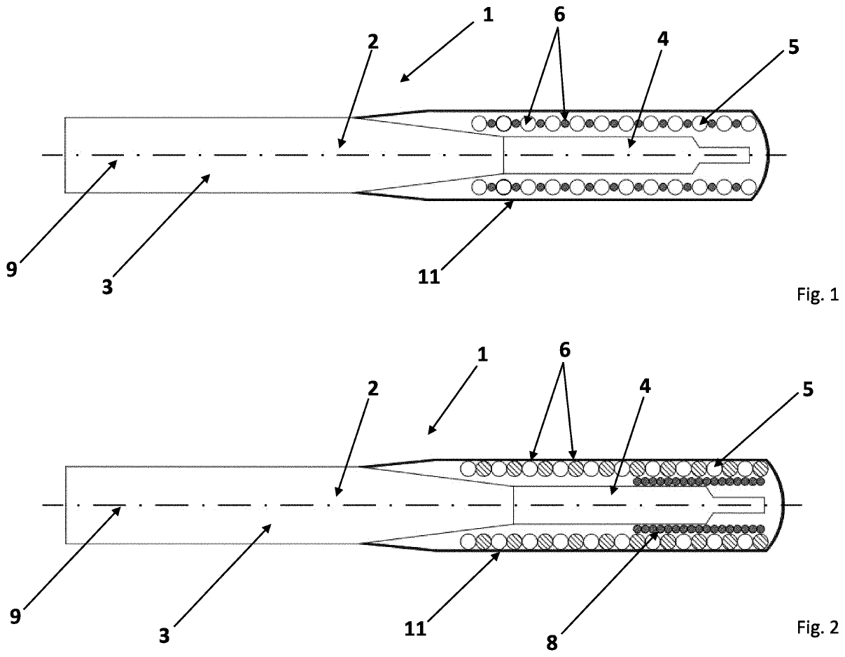

FIG. 1 shows a side view of the guide wire according to the invention according to a first embodiment;

FIG. 2 shows a side view of the guide wire according to the invention according to a second embodiment;

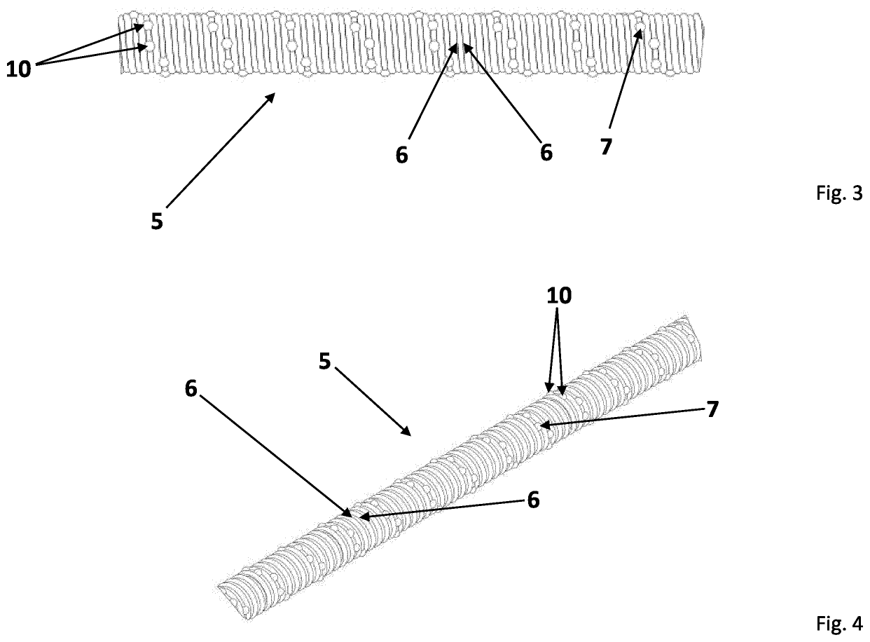

FIG. 3 shows a side view of a part of a wire coil of the guide wire according to the invention according to a further embodiment;

FIG. 4 shows a diagonal view of the part of the wire coil shown in FIG. 3;

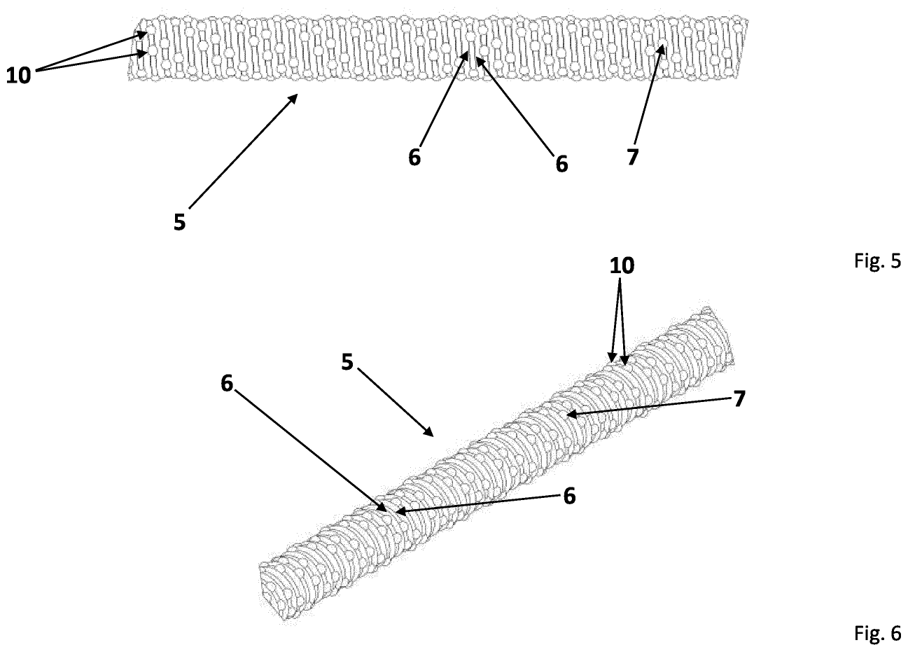

FIG. 5 shows a side view of a part of a wire coil of the guide wire according to the invention according to a further embodiment; and

FIG. 6 shows a diagonal view of the part of the wire coil shown in FIG. 5.

FIG. 1 shows a side view of a guide wire 1 according to the invention, with a distal portion 4 adjoining a proximal portion 3 along the longitudinal axis 9. The length of the proximal portion 3, which is not shown in full here, clearly exceeds the length of the distal portion 4. The guide wire 1 has a core wire 2 the diameter of which decreases in the distal portion 4. In the distal area of the core wire 2, a wire coil 5 is fixed thereto such that the total diameter of guide wire 1 in the distal portion 4 corresponds approximately to that in the proximal portion 3. The proximal portion 3 of the core wire 2 is made of a cobalt-chromium-nickel-molybdenum alloy and the distal portion 4 is made of a pseudo-elastic nickel-titanium alloy. The distal portion 4 of guide wire 1 is provided with a hydrophilic coating 11 on the outside.

The wire coil 5 is composed of two wires 6 running alongside each other in the form of a spiral, wherein wires 6 with different diameters are used in this exemplary embodiment. The thicker wire 6 is made of a nickel-titanium alloy, the thinner wire 6 is a platinum wire, which provides the radiopacity of the distal portion 4.

FIG. 2 shows a variant of the guide wire 1 according to the invention, which corresponds to the basic structure of FIG. 1. However, the wire coil 5 here is composed of two wires 6 with identical diameters, with one wire 6 again being made of a nickel-titanium alloy, while the other wire 6 is a DFT wire with a radiopaque interior made of platinum or a platinum alloy and a pseudoelastic sheath made of nickel-titanium. In addition, a further wire coil 8 is provided inside the wire coil 5, which only extends over part of the length of the wire coil 5 and is made of platinum.

FIG. 3 shows only part of a wire coil 5, namely the distal part. The wires 6 running alongside each other in the form of a spiral are each connected to each other at connecting points 7 arranged in pairs. The offset within each pair of connection points 10 is 45°. The next pair of connection points 10 is arranged at an offset of 440° to the previous pair of connection points 10. Since the offset between the two connection points 7 in this pair of connection points 10 is also 45°, this means that the offset between the first connection points 7 of two consecutive pairs of connection points 10 is also 440°. The same applies to the offset between the two second connection points 7 of two consecutive pairs of connection points 10. The connection points 7 are usually welding points.

Overall, there are relatively large distances between the pairs of connection points 10 in this embodiment, i.e. the total number of connection points 7 is relatively small. This results in a high level of flexibility of the wire coil 5, which is particularly desirable in the distal area.

FIG. 4 shows a diagonal view of the wire coil from FIG. 3. You can see the comparatively loose distribution of the connection points 7.

FIG. 5 shows another part of a wire coil 5, which is more likely to be found in the proximal area of the wire coil 5. Here too, the offset between the connection points 7 of a pair of connection points 10 is 45°, but the offset between the pairs of connection points 10 is much smaller at 130°. Correspondingly, the connection points 7 are arranged closer together, which is associated with a lower level of flexibility, but higher rigidity and better torque transmission.

FIG. 6 shows a diagonal view of the wire coil from FIG. 5. You can see the comparatively dense distribution of the connection points 7.

1. A guide wire with a core wire having a proximal portion and a distal portion, wherein the distal portion of the core wire is surrounded by at least one wire coil,
characterized in that,
the at least one wire coil is composed of at least two individual wires, which run alongside each other in the form of a spiral, and wherein the at least two wires are connected to each other at multiple connection points along the at least one wire coil.

2. The guide wire according to claim 1, characterized in that the at least two wires are made of at least two different materials.

3. The guide wire according to claim 1, characterized in that the core wire has a smaller outer diameter in the distal portion than in the proximal portion.

4. The guide wire according to claim 1, characterized in that the multiple connection points are welding points.

5. The guide wire according to claim 1, characterized in that the number of connection points along the at least one wire coil decreases from proximal portion to distal portion of the core wire.

6. (canceled)

7. The guide wire according to claim 1, characterized in that the offset between a first connection point and a second connection point distal to the first connection point connection is approximately 180°.

8. The guide wire according to claim 7, characterized in that the offset between the second connection point and a third connection point distal to the second connection point is 15° to 90°.

9. (canceled)

10. (canceled)

11. (canceled)

12. The guide wire according to claim 1, characterized in that at least one of the wires forming the at least one wire coil is radiopaque.

13. (canceled)

14. The guide wire according to claim 1, characterized in that a pitch of the at least one wire coil (5) decreases from proximal to distal.

15. (canceled)

16. (canceled)

17. (canceled)

18. (canceled)

19. The guide wire according to claim 1, characterized in that at least one of the wires forming the at least one wire coil are twisted.

20. The guide wire according to claim 1, characterized in that the distal portion of the core wire is surrounded by two wire coils arranged radially one above the other.

21. (canceled)

22. Guide wire comprises a core wire having a proximal section and a distal section, wherein the distal section of the core wire is surrounded by at least one wire coil formed from at least two individual wires running spirally next to each other, wherein the at least two wires are connected at a plurality pairs of connection points, and wherein an offset between a proximal connection point and a distal connection point of a same pairs of the connection points is 30° to 60°.

23. Guide wire according to claim 22, characterized in that an offset between the respective proximal connection points of two adjacent pairs of the connection points is 130° to 440°.

24. Guide wire according to claim 22, characterized in that the proximal connection point of a first pair of connection points offsets the respective proximal connection point of an adjacent pair of connection points distal to the first pair of connection points by a first degree, and the proximal connection point of the first pair of connection points offsets the respective proximal connection point of an adjacent pair of connection points proximal to the first pair of connection points by a second degree; and wherein the first degree is greater than the second degree.

25. Guide wire according to claim 22, characterized in that two wire coils surround the distal section of the core wire with a first wire coil wounding over the distal section of the core wire and a second wire coil wounding over the second wire coil.

26. Guide wire according to claim 25, characterized in that the second wire coil with at least two individual wires running spirally next to each other is radially above the first wire coil.
